(19) United States
(12) Patent Application Publication
(10) Pub. No.: US 2014/0350352 A1
(43) Pub. Date: Nov. 27, 2014

(54) SYSTEM AND METHOD OF ASSESSING STABILITY OF PATIENTS

(71) Applicant: Children's Medical Center Corporation, Boston, MA (US)

(72) Inventors: Dimitar Valeriev Baronov, Boston, MA (US); Evan James Butler, Boston, MA (US); Peter Laussen, Newton, MA (US); Melvin Almodovar, South Natick, MA (US)

(73) Assignee: CHILDREN'S MEDICAL CENTER CORPORATION, Boston, MA (US)

(21) Appl. No.: 13/900,992

(22) Filed: May 23, 2013

(51) Int. Cl. A61B 5/00

(52) U.S. Cl. CPC A61B 5/7275
USPC 600/301

(57) ABSTRACT

In one embodiment, a technique for assessing a stability of a patient is provided. In particular, a plurality of estimated trends for each physiological variable of a patient are calculated by utilizing a plurality of physiological variables from a plurality of medical devices and a plurality of target ranges for each physiological variable. These trends are then used to calculate dynamically a single stability value over time of the patient based the target ranges input by a user for each physiological variable in relation to the estimated trends. In particular, this stability value a single value that accounts for and reflects the stability/acuity of a plurality of physiological variables to indicate quickly to physicians the stability of the patient.

### TECHNICAL FIELD

The present disclosure relates generally to a system and method for dynamically synthesizing multiple physiological variables of a patient received from a plurality of medical devices and reducing these physiological variables into a single stability value based on that physiological data (variables) received in relation to the patient to assess a patient's current stability, i.e. the ability to maintain homeostasis.

### BACKGROUND

In a hospital patients are constantly being monitored by physicians to determine the stability of the patient. Physicians use data collected from a plurality of different medical devices such as ventilators, heart rate monitors, blood pressure monitors, brain tissue oxygen saturation monitors, temperature sensors, etc., to determine and assess whether or not the patient is stable enough to be moved in to a non-critical facility. The medical devices is attached to a patient continuously collect data which is then displayed on a monitor, typically at the patients beside. Physicians making their rounds in then use this information to determine the stability of the patient and the likelihood that patient is going to lapse back into a serious condition.

The clinical concept of stability is associated with an ability of a patient's physiology to function efficiently and sustain life. As a result, the physiology, as a stable dynamic system, regulates the physiologic variables within targeted ranges (or bounds) that assure efficient body operation. However, once the physiology loses this ability to regulate these variables within these bounds, a patient is thought to have become unstable.

In particular, the concept of stability is of particular importance in an Intensive Care Unit (ICU), also known as a Critical Care Unit (CCU), Intensive Therapy Unit or Intensive Treatment Unit (ITU). An ICU is special department of a hospital or health care facility that provides intensive-care to patients of all ages. ICUs may also be made up of specialized units such as, the Pediatric Intensive Care Unit (PICU), the Cardiac Intensive Care Unit (CICU), the Newborn Intensive Care Unit (NICU), etc.

Thus, ICUs typically cater to patients with the most severe and life-threatening illnesses and injuries in the hospital. These injuries or illnesses typically require constant, close monitoring and support from physicians, specialized equipment (like medical devices described above) and medication in order to maintain “normal” bodily functions in hopefully a stable state. Common conditions that are treated within ICU's include those such as trauma, organ failure, sepsis, etc.

Currently in an (ICU) setting, physicians are required to monitor a large number of patients and make quick determinations regarding the impending stability or the overall health of a patient. These determinations are often based on the data that is being collected from the plurality of specialized medical devices that are attached to the patient and is displayed on a screen along the patient's bedside. As one can imagine, the monitors that display this data include a plethora of data which the physicians must interpolate and reference, quickly and definitively. This can be lead to a certain inefficiency in the ICU, which typically should be operated as efficiently as possible to ensure there is no loss of life.

### SUMMARY

In one embodiment, a technique for assessing a stability of a patient is provided which dynamically assess the stability of a patient and provides physicians with a single value that reflects an analysis of a plethora of physiological variables provided by numerous medical devices that are attached to a particular patient. In particular, a plurality of estimated trends for each physiological variable of a patient may be calculated by utilizing the plurality of physiological variables that have been received from the plurality of medical devices and a plurality of target ranges for each physiological variable. These trends are then used to calculate dynamically a single stability value related to the patient based the target ranges of each physiological variable in relation to the estimated trends. This stability value is a single value that accounts for and reflects the plurality of physiological variables and each variables associated trend to indicate the stability or acuity of the patient as a single value which the physician can use to assess the stability of the patient quickly and efficiently, thus being able to more quickly identify those patients that are the least stable.

Furthermore, in some exemplary embodiments of the present invention, the estimated trend of each physiological variable may be calculated by performing a plurality of linear estimations over a number of time intervals for each physiological variable associated with the patient. The linear estimation may be performed for each signal that is received from a particular medical device over a first period of time that is subsequently parsed into a second period of time that is smaller than the first period of time.

The second period of time may be parsed additively and inclusively. In particular, the length of time associated with the first period of time may determine how much of the patients history is included in the linear estimation, while the length of time of the second period of time, however, may determine how sensitive the stability value will be to resulting trends. Both the first period of time and the second period of time may be set prior to performing linear estimations and may be based at least on an input by a user and physiologic process time constraints. These estimations may then be used to calculate the single stability value which represents the stability/acuity of the patient.

Furthermore, in some exemplary embodiments, when the stability of the patient is initially assessed before a certain amount of data and time values has been acquired, a third time period set back from a current time value may be generated. In particular, the linear estimation in this instance is performed over the third time period, and once a sufficient amount of data and time values has been acquired each time value may then be looped accordingly. As a result, within each of these loops a dominate trend, a standard deviation and an estimated slope may be calculated for each physiological variable.

From here the stability value may be calculated in a number of ways. For example, for each physiological variable over a period of time, the trend calculation may return an estimated slope, a standard deviation and an estimation value of each physiological variable over a given period of time. An utility map estimate for each physiological variable by may then be calculated by feeding the estimated trend of each physiological variable at the given point in time through a utility map to normalize each physiological variable relative to other physiological variables of the plurality of physiological variables and weight each of the normalized physiological variables depending on where each of the normalized physiological variables falls within the targeted range of that physiological variable. This targeted range for each physiological variable may be set by a user (e.g., the physician) or may be determined dynamically by the system based on experimental data or standards. Alternatively, utility map data for each physiological variable may output prior to calculating estimated trends.

Regardless, in some exemplary embodiments, one way single stability value may be calculated is by multiplying a mean of the utility map estimate of each physiological variable by an estimated slope of each physiological variable to output a mean value for each physiological variable. The exemplary technique may then sum of the mean value for each physiological variable over the plurality of physiological variables.

In other exemplary embodiments of the present invention, another way the single stability value may be calculated is by multiplying a mean of the utility map estimates of each physiological variable by the estimated slope of each physiological variable to output a max value for each physiological variable and summing the max value for each physiological variable over time.

In yet other exemplary embodiments of the present invention, the single stability value may be calculated by multiplying a mean of the utility map estimates of each physiological variable by the estimated slope of each physiological variable to output a resulting mean value, then multiplying the output resulting mean value by a normalized standard deviation value to output a weighted value for each physiological value, and summing the weighted value for each physiological value over the plurality of physiological variables.

Even further in yet other exemplary embodiments of the present invention the single stability value is calculated by multiplying a mean of the utility map estimates of each physiological variable by the estimated slope of each physiological variable to output a resulting mean value, then multiplying the outputted resulting mean value by a length of time associated with each trend to output a weighted value, and summing the weighted value over the plurality of physiological variables.

Advantageously, the exemplary embodiments of the present invention provides physicians with a single stability value acuity value) that takes into account an aggregate of physiological variables that have been collected by a plurality of medical devices, such as ventilators, heart rate monitors, blood pressure monitors, brain tissue oxygen saturation monitors, temperature sensors, etc. That is, the illustrative embodiment of the present disclosure is able to listen a hospital's infrastructure and data that is being collected and calculate a single value (i.e., a stability index value) that a physician can use to better assess which patients are at the highest risk of having a dangerous condition occur (i.e., which patients are the most acute).

The additional features of the present disclosure will be described infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates an example communication network for communicating and processing the data;

FIG. 2 illustrates an example network device/node on which the stability value may be processed;

FIG. 3 illustrates an example view of techniques for calculating the stability value;

FIG. 4 illustrates one exemplary embodiment for calculating the stability value from the estimated trend data; and

FIG. 5 illustrates another exemplary embodiment for calculating the stability value from the estimated trend data.

### DESCRIPTION OF EXEMPLARY EMBODIMENTS

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Furthermore, the control logic of the present invention may be embodied as non-transitory computer readable media on a computer readable medium containing executable program instructions executed by a processor, controller or the like. Examples of the is computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

Illustratively, the techniques described herein are performed by hardware, software, and/or firmware, which may contain computer executable instructions executed by the processor 220 (or independent processor of interfaces 210) to perform functions relating to the techniques described herein, e.g., in conjunction with communication process 244. For example, the techniques herein may executed on an aggregate of servers over wireless communication protocols, and as such, may be processed by similar components understood in the art that execute those protocols, accordingly.

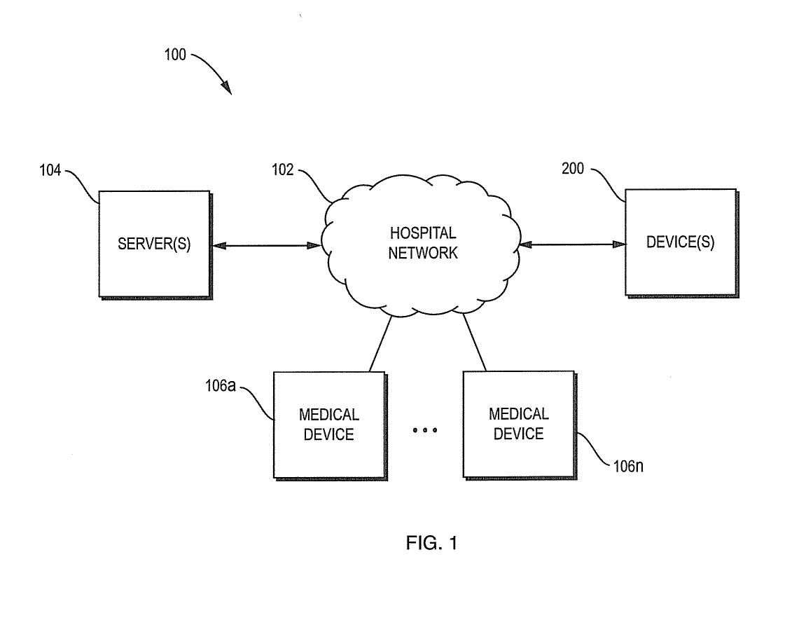

FIG. 1 is a schematic block diagram of an example hospital network 100 illustratively comprising nodes/devices 200 (e.g., computers, mobile devices, or any other computational device that is capable of displaying an interactive interface) interconnected by various methods of communication. For instance, communication between the devices may be by wired links or by a wireless communication medium, where certain devices or node 200 may be in communication with other devices or nodes 200, e.g., based on distance, signal strength, current operational status, location, etc. Those skilled in the art will understand that any number of nodes, devices, links, etc. may be used in the computer network, and that the view shown herein is for simplicity. Furthermore, Also, the device(s) 200 may be connected over a private or public network 102 to one or more servers 104 which collect data from one or more medical devices 106a-n (e.g. ventilators, heart rate monitors, blood pressure monitors, brain tissue oxygen saturation monitors, temperature sensors, etc.)

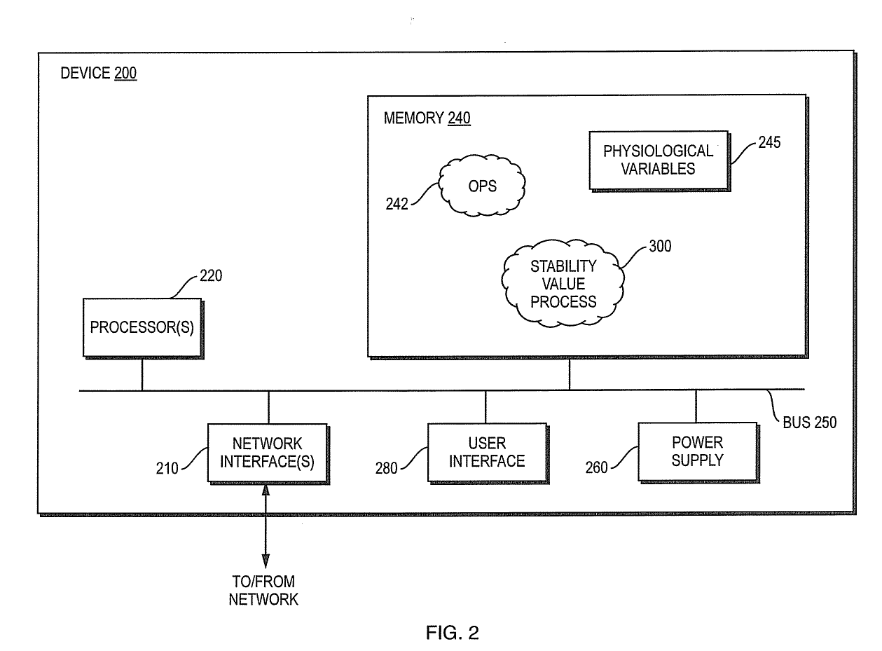

FIG. 2 is a schematic block diagram of an example node/device 200 that may be used with one or more embodiments described herein, e.g., as an interfacing device in the network. The device may include one or more network interfaces 210, one or more user interfaces 280, at least one processor 220, and a memory 240 interconnected by a system bus 250, as well as a power supply 260 (e.g., battery, plug-in, etc.).

The network interface(s) 210 contain(s) the mechanical, electrical, and signaling is circuitry for communicating data over physical and/or wireless links coupled to the network 102. The network interfaces may be configured to transmit and/or receive data using a variety of different communication protocols, including, inter alia, TCP/IP, UDP, wireless protocols (e.g., IEEE Std. 802.15.4, WiFi, Bluetooth®), Ethernet, power line communication (PLC) protocols, etc. Namely, one or more interfaces may be used to communicate with the user on multiple devices and these interfaces may be synchronized using known synchronization techniques.

The memory 240 may include a plurality of storage locations that are addressable by the processor 220 and the network interfaces 210 for storing software programs and data structures associated with the exemplary embodiments described herein. As noted above, certain devices may have limited memory or no memory (e.g., no memory for storage other than for programs/processes operating on the device). The processor 220 may comprise necessary elements or logic configured to execute the software programs and manipulate the data structures, such as physiological variables 245. An operating system (OPS) 242, portions of which are typically resident in memory 240 and executed by the processor, functionally organizes the device by, inter alia, invoking operations in support of software processes and/or services executing on the device.

These software processes and/or services may include the exemplary stability index process 300 as described below, which may include linear estimation trend processes, good fit calculation processes, weighting processes, etc. It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules configured to operate in accordance with the techniques herein (e.g., according to the functionality of a similar process).

The stability index process 300 may contain computer executable instructions executed by the processor 220 to perform calculations related to a stability value of a patient. These functions may be performed on either the device 200 or on the server 104. Should the process be performed on the server, those skilled in the art will understand that the device 200 receives the output values through the network interface(s) 210 and displays them to a user via the user interface 280 on a screen. Additionally, data input by the user (such as target ranges) may be communicated to the server 104 in this embodiment as well.

### The Stability Index Process

More specifically, the purpose of the stability value is to evaluate the acuity/stability of the patient's physiology in a manner consistent with systems theory. In systems theory, an important characteristic of stability is the system's attracting set, which describes what is the furthest that a system can be perturbed from its equilibrium point and still return back to the equilibrium point. In this set, attracting forces are counteracting the disturbances, and by describing the magnitude of these forces, one can fully describe the stability of the system. In the exemplary embodiment of the present invention, the attracting forces are described by the utility functions, which implement medical knowledge or targeted trends to define how strong the physiology is expected to drive the variables back to their optimal values, given how far away the physiological variables are from these values. This definition provides the ability to analyze whether there exist destabilizing forces and how hard they are acting against the stability enforced by the body's automated regulation and the therapy provided by the physician.

As stated above, in an (ICU) setting, physicians are required to monitor a large number of patients and make quick determinations regarding the impending stability or the overall health of a patient. These determinations are often based on the data that is being collected from the plurality of specialized medical devices that are attached to the patient and displayed on a screen along the patient's bedside. As one can imagine, the monitors that display this data include a plethora of data which the physicians must interpolate and reference, quickly and definitively. This can be lead to a certain inefficiency in the ICU, which typically should be operated as efficiently as possible to ensure there is no loss of life.

Hospitals have begun to store the data collected by each of these medical devices on a server so that the physicians can look back at the data and analyze the data over time for particular patient. However, there is currently no technique for analyzing and assessing dynamically this data in real time to provide, physicians, who have often little time to make is an assessment regarding the patient's stability.

Therefore, the exemplary embodiment of the present invention provides a technique for assessing a stability of a patient is provided which dynamically assess the stability of a patient and provides physicians with a single value that reflects an analysis of a plethora of physiological variables provided by numerous medical devices that are attached to a particular patient. In particular, a plurality of estimated trends for each physiological variable of a patient may be calculated by utilizing the plurality of physiological variables that have been received from the plurality of medical devices and a plurality of target ranges for each physiological variable. These trends are then used to calculate dynamically a single stability value related to the patient based the target ranges of each physiological variable in relation to the estimated trends. This stability value is a single value that accounts for and reflects the plurality of physiological variables and each variables associated trend to indicate the stability or acuity of the patient as a single value which the physician can use to assess the stability of the patient quickly and efficiently, thus being able to more quickly identify those patients that are the least stable.

A more detailed description will now be described.

The stability index process 300 may be embodied as an algorithm that dynamically synthesizes multiple physiologic variables and outputs a single value that assesses a patient's stability/acuity.

As a high level description of the stability index process 300, the physiological variables/signals from numerous devices 106a-n (n being an infinite value) over iterated time scales are acquired/received, parsed, and filtered by the system. For the purposes of this document, the above acquisition, parsing and filtering will not be described since this portion of the process is platform specific and would be well understood by those skilled in the art. Once the data is acquired, parsed and filtered by patient and targeted ranges are input, estimated trends may then be calculated based linear estimation and goodness of fit calculations of signals/physiological variables from the medical devices over a number of inclusive time intervals. Then based on physician set target ranges for each parameter, a single stability value may be output which summarizes the stability of a patient.

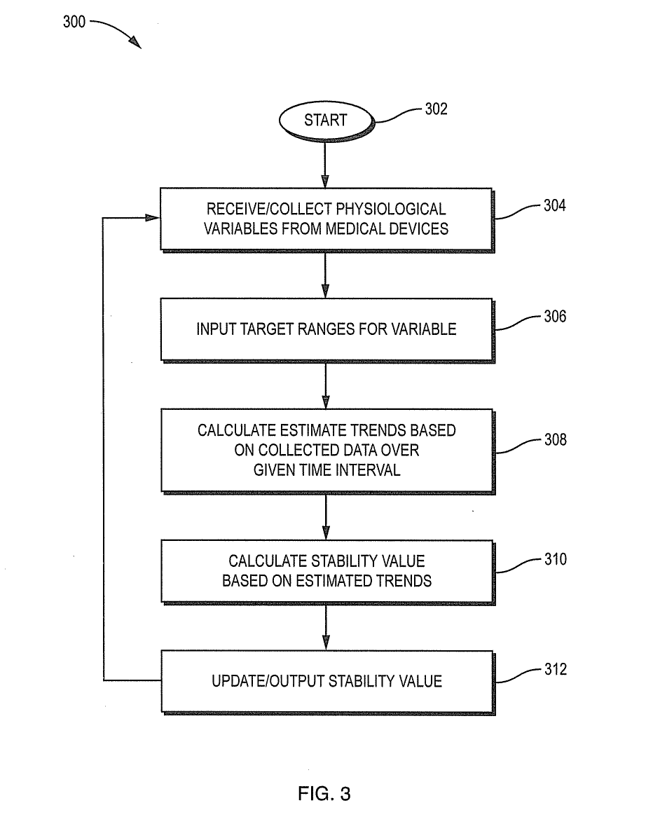

Operationally, for example, with reference to FIG. 3, the stability index process 300 starts at step 302 upon the initial collection/reception of a physiological variables (i.e., data/signals for each of the physiological variable) associated with a particular patient from numerous medical devices (e.g., heart rate monitor, ventilator, brain oxygen content monitor, blood pressure monitor, etc.). The physiological variables are received/collected, parsed and filtered over numerous time scales from numerous medical devices in step 304. Also initially, via the user interface(s) 280 the physicians also may enter a targeted range for each of the physiological variables in step 306. These targeted ranges refer to the ideal range for each variable which may or may not vary from patient to patient based on information known regarding physiological cause and effects. Alternatively, the targeted ranges may be may be determined dynamically by the system based on experimental data or standards none in the medical field.

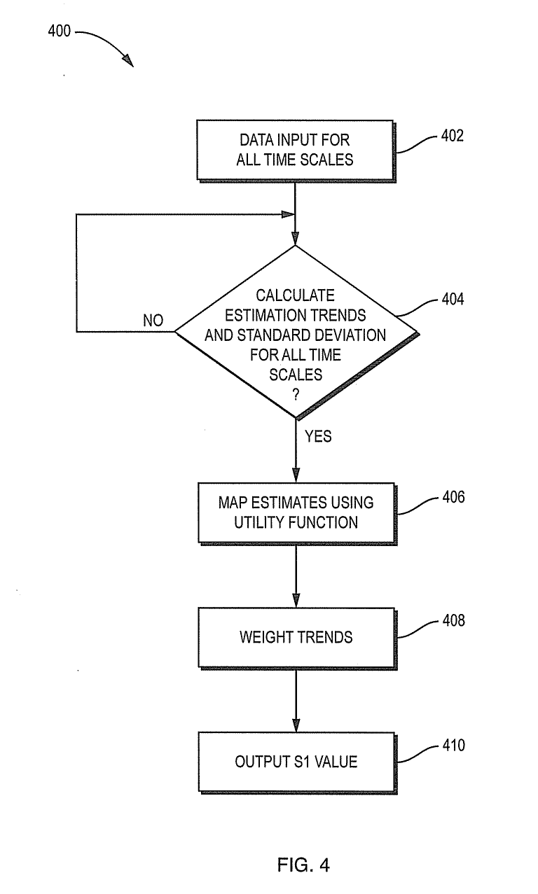

Next, utilizing the physiological variables, estimated trends for each physiological variable may be calculated via, e.g., linear estimations and goodness of fit calculations in step 308. These trends may be mapped in some embodiments like the one shown in FIG. 4 in which once all of the time scales are calculated the trends are mapped using various utility functions described below. Using these mapped targeted trends and/or a standard deviation value, a single stability value related to the patient based the target ranges of each physiological variable may be dynamically calculated and output to the physicians via the user interface either in numerical or graphical form in step 310, and a single stability value that reflects and accounts for the plurality of physiological variables as a whole to indicate an acuity level or stability value of the patient is output in step 312.

More specifically, in some exemplary embodiments, initially, when the stability of the patient is assessed, there are not enough data and time values to calculate an estimated trend. In this situation a time period back from a current time value may be generated by the system and a trend may be estimated over this period of time which is smaller than the period of time that system is configured to used. As a result the stability value is calculated using smaller period of time. However, once a sufficient amount of data and time values has been acquired, each time value is looped and within each loop a dominate trend (y), a standard deviation (σ) and an estimated slope (b) is calculated for each physiological variable.

To estimate a trend, a linear estimation may be performed for each signal received from a particular medical device over a first period of time that may be subsequently parsed into a second period of time that is smaller than the first period of time. Linear estimates may be made for each individual physiological variable from each medical device over a first period (Tₜ) that is subsequently parsed into smaller second time period (Tₜ). The parsing may be done additively and inclusively, so for example when a Tₜ of 3 hours is chosen with a Tᵢ of 1 hour the time windows over which the trend estimation would be made may be, for example:

Iteration 1: data from (Lₙ−1 hour):tₙ

Iteration 2: data from (Lₙ−2 hours):tₙ

Iteration 3: data from (Lₙ−3 hours):tₙ

In the above iterations, is equal to the current time value, and the size of Tₜ determines how much of the patient history is included, and size of Tᵢ determines how sensitive it will be to emerging trends. For instance a larger Tᵢ would discount fast trends because those trends would essentially be filtered out by the other data in that Tᵢ window.

For example, if Tᵢ is 5 minutes instead of an hour, 36 trend estimation calculations would have been made instead of 3. Modifications in some exemplary embodiments can be made to have variable step scale (e.g., Tᵢ may be 1 minute for the first few steps but is then increased to a larger number over time), but for ease of understanding the exemplary embodiment will be described as a fixed value in the present disclosure.

That is, the second period of time may be parsed additively and inclusively and a length of time associated with the first period of time is used to determine how much of the patients history is included in the linear estimation, and a length of time of the second period of time determines how sensitive the stability value will be to resulting trends. Preferably, the first period of time and the second period of time may be set prior to performing linear estimations via the user interface 280 and physiologic process time constraints.

Determinations of the values of Tₜ and Tᵢ may be made prior to running the stability index process 300, based on user input and trend durations of interest by physicians, physiologic process time constants (e.g., how quickly the human body can actually change) and computational constraints (e.g., how many iterations can be run over a given time period).

As stated above, the first step in the stability index may be to generate the durations back from the current time step (Lₙ) over which the estimation will be performed tₛₜₑₚₛ. That is, as stated above, initially there may not be enough data (i.e., physiological variables) present to perform the trend analysis over the full Tₜ duration so the actual time scale is determined dynamically via for example using the following algorithm:

if tₙ-Tₜ < t0
window = tₙ-t₀
else
window = Tₜ
increments = Tᵢ to window in steps of Tᵢ
tₛₜₑₚₛ = tₙ-increments

Where tₒ is the zero time associated with the physiological variables (e.g., signals from the medical devices). Once tₛₜₑₚₛ has been established each value is looped and within that loop a slope and standard deviation (σ) is calculated for each physiological variable. For example, the following algorithm may be utilized:

```
for t in tₛₜₑₚₛ
    for signal in signals
        partial signal = signal(tₙ-t to tₙ)
        [slope, σ, Xbar ] = estimate trend function (partial signal)
```

Xbar may be understood as an estimation of the final data point according to the linear trend estimated by the estimate trend function. The estimate trend function operates by performing principle component analysis of the data after it has been modified to be a zero mean. Accordingly, when x is the data and t is the associated time vector:

t=t−mean(t)

x=x−mean(x)

Both the time and the data zero mean series may be made and the dominant eigenvector is extracted from a modified data covariance as follows:

[eigenvectors,eigenvalues]=eig(x*x′)

The returned values may be paired; take the eigenvector (V) associated with largest eigenvalue. Next, the dominant trend may be derived and the data may be projected on to the resulting trend in:

Y=x*V

Finally, the slope (b) may be estimated using, for example, a goodness of fit calculation:

b=Y′*t/(t*t′)

From this the standard deviation of the trend (σ) can be calculated:

residual=Y−b*t

A heuristic degrees of freedom (DF) equivalent can then be generated by observing the residuals volatility about zero. Leading to:

σ=square root(R*R′/t*t′/DF)

Thus, for each physiological variable of the plurality of physiological variables over a period of time, the trend calculation returns an estimated slope b, a standard deviation σ and an estimation value of each physiological variable at over a given period of time tₙ (Xbar).

As stated above, a utility map estimate for each physiological variable may be output by feeding the estimated trend of each physiological variable at the given point in time through a utility map to normalize each physiological variable relative to other physiological variables. Then each of the normalized physiological variables may be weighted depending on where each of the normalized physiological variables falls within a targeted range of that physiological variable that has been previously set by a physician.

More specifically, as shown in FIG. 4, the Xbar value above may be feed through a utility map, which essentially normalizes the various physiologic parameters relative to each other, and weights them depending on where the values fall relative to the range of expected values. The output from this is referenced herein as Ybar. Thus, in this method 400, the data is input for all time scales in step 402, then estimated trends and standard deviations for each trend are calculated for all time scales for the physiological variables in step 404 and iterated until all values are estimated. Then once the estimated trends for each physiological variable is calculated, a utility map of the estimated trends is generated in step 406, the estimated trends are weighted in step 408 and then a stability value is output in step 410.

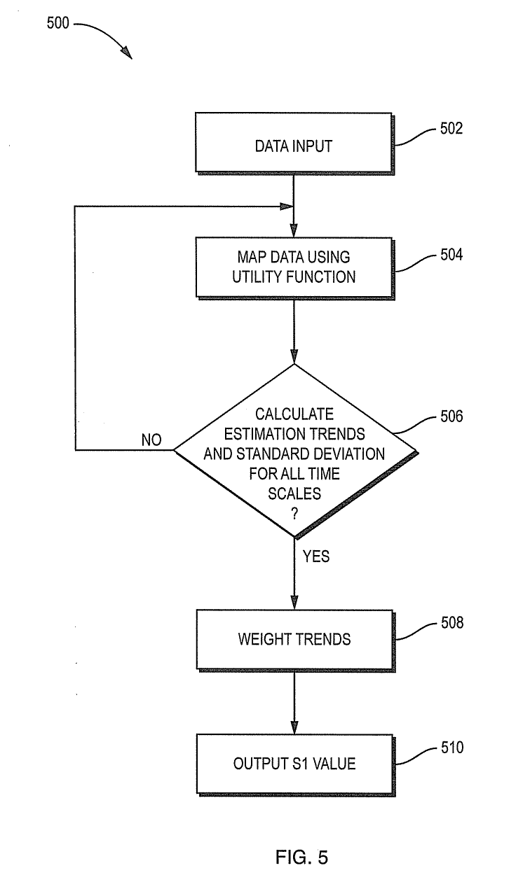

Alternatively, FIG. 5 illustrates another exemplary embodiment for calculating the stability index from the estimated trends. In this exemplary embodiment, utility map data for each physiological variable is output prior to calculating estimated trends. In particular, in this embodiment, the data is input for all time scales in step 502, then, in this embodiment the is data is mapped prior to estimating the trend in step 504. Then once the estimated trends for each physiological variable is calculated, the estimated trends are weighted in step 508 and then a stability value is output in step 510.

More specifically, in this embodiment the slope of the physiological variable is estimate before trending is calculated, i.e. raw data is first normalized and weighted and then a slope (b) and a standard deviation (σ) for each physiological variable may be estimated. In particular, this may be accomplished by passing partial signal array through the utility map function prior to being passed on to an estimated trend function. In this case, Ybar may be a matrix populated with 1 values, because all of the utility function information may be included in the slope b and standard deviation values.

Once estimated trends and their associated standard deviations have been estimated the stability value can be calculated. More specifically, the stability value may be calculated by using the mapped estimates to determine a mean value, a max value, a weighted mean standard deviation value, or a weighted mean time. However, these methods are merely exemplary and may be calculated via other means.

Mean Value Calculation

For example, when the stability value is calculated via a ‘mean’ calculation, the single stability value may be calculated by multiplying a mean of the utility map estimates of each physiological variable by an estimated slope (b) of each physiological variable to output a mean value for each physiological variable and a sum of the mean value for each physiological variable over the plurality of physiological variables. Thus, utilizing the following equation:

Stability value=sum over parameters(mean(abs(Ybar).*abs(b)))

In the above method, for example, when a single physiological variable has a desired range of 50-100. One of the time scales returns an Xbar of 120 with the estimated slope (b) value of 2. When Xbar is passed through an utility map, Ybar is returned as a larger value because Xbar would be well outside of a desired target range. Additionally, with a slope (b) of 2, it may be clear that there is a strong trend. Given these two factors; outside of range and a strong trend, the algorithm will return a higher stability value (e.g., 4, where 0 is the lowest and 4 is the highest).

On the contrary, when the same scenario as above is applied for a given patient, but the slope (b) is calculated to be 1×10⁻⁵. The Ybar will again be large, but since the Ybar, in this instance, is not paired with a substantive estimated trend this instance will return a smaller stability value (e.g., 3). Thus, in this example, even though the patient is well outside a targeted range, the patient may be assessed as stable, because the physiological variable is not changing. Thus, there may be a number of gradations between two extreme scenarios. Therefore, outcome studies may be performed to properly associate the stability value outputs with meaningful clinical outcomes.

Max Value Calculation

Alternatively, in other exemplary embodiments of the present invention, the stability value may be calculated using a ‘max value’. In this embodiment again a single stability value may be calculated by multiplying a mean of the utility map estimates of each physiological variable by an estimated slope (b) of each physiological variable to output a max value for each physiological variable and summing the max value for each physiological variable over time.

Stability Value=max(sum over time(abs(Ybar).*abs(b)))

Here, instead of summing the max value over the physiological variables, the max values are summed over time. So, each time scale has an associated value, the maximum of which is then reported as the stability value.

Weighted Mean Standard Deviation Calculation

Even further, the in the stability value may also be calculated by a ‘weighted mean standard deviation which takes into account the standard deviation associated with each trend estimate. Again, the calculation starts by multiplying a mean of the utility map estimates of each physiological variable by an estimated slope of each physiological variable to output a resulting mean value. Then the output resulting means value is multiplied by a normalized standard deviation value to output a weighted value for each physiological value, and the weighted value is summed for each physiological value over the plurality of physiological variables.

In particular, again the following equation may be applied:

y=abs(Ybar).*abs(b)

This is then weighted using a normalized σ value, and all of the values are summed to produce an stability value output:

w=1/σ

y_{w}=y*w

Stability Value=Sum over both physiological variables and time(y*1/w)

This method works by attributing more weight to trends that have lower standard deviations.

Weighted Mean Time Calculation

Additionally, the stability value can also be calculated by a utilizing a ‘weighted mean time,’ whcih is similar to using a ‘weighted mean standard deviation, but instead of using the standard deviation, a length of each trend is used to weight the estimated trend. Therefore, again, a mean of the utility map estimates of each physiological variable is multiplied by an estimated slope of each physiological variable to output a resulting mean value. This outputted resulting mean value is then, however, multiplied by a length of time associated with each trend to output a weighted value, and the weighted value is summed over the plurality of physiological variables.

Using this method, w is equal to a number of increments. In this exemplary embodiment of the present invention, increments may be defined as an array containing all time scales over which trends may be estimated. Again, the following may be applied:

y=abs(Ybar).*abs(b)

However, as stated above, w=increments

y_{w}=y.*w

Stability value=sum over both physiological variables and time(y*1/w)

As a result of this calculation, trends that have been occurring for a longer period of time are weighted more heavily than those trends that occur for shorter periods of time.

However, regardless of which calculation is used to calculate the stability value, the final output value should reflect to physicians a value that reflects a patient's stability and acuity. The above stability value may range for example from 1-4, however, the illustrative embodiment of the present invention should not be limited as such and may in alternative embodiments be embodied as ranging from 1-10.

Advantageously, the exemplary embodiments of the present invention provides physicians with a single stability value acuity value) that takes into account an aggregate of physiological variables that have been collected by a plurality of medical devices, such as ventilators, heart rate monitors, blood pressure monitors, brain tissue oxygen saturation monitors, temperature sensors, etc. That is, the illustrative embodiment of the present disclosure is able to listen a hospital's infrastructure and data that is being collected and calculate a single value (i.e., a stability index value) that a physician can use to better assess which patients are at the highest risk of having a dangerous condition occur (i.e., which patients are the most acute).

While there have been shown and described illustrative embodiments that specific calculations for calculating the above stability value (index), those skilled in the art will understand than there may be other ways to calculate the above trends and singular output value, thus the illustrative embodiment of the present invention should not be limited as such. Furthermore, although some medical devices have been provided, the illustrative embodiment of the present invention can utilize data from any number of medical devices and may be displayed on any number of computerized devices, such as mobile phone, smartphone, computer, laptop computer, etc. Also, although the above technique has been described as being processed in a particular order, the illustrative embodiment is not necessarily limited as such since.

The foregoing description has been directed to specific embodiments. It will be apparent, however, that other variations and modifications may be made to the described embodiments, with the attainment of some or all of their advantages. For instance, it is expressly contemplated that the components and/or elements described herein can be is implemented as software being stored on a tangible (non-transitory) computer-readable medium (e.g., disks/CDs/RAM/EEPROM/etc.) having program instructions executing on a computer, hardware, firmware, or a combination thereof. Accordingly this description is to be taken only by way of example and not to otherwise limit the scope of the embodiments herein. Therefore, it is the object of the appended claims to cover all such variations and modifications as come within the true spirit and scope of the embodiments herein.

What is claimed is:

1. A method for assessing a stability of a patient, comprising:
receiving, at a processor, a plurality of physiological variables from a plurality of medical devices;
receiving, at the processor, a plurality of target ranges for each physiological variable of the plurality of physiological variables;
calculating, by the processor, a plurality of estimated trends for each physiological variable over time;
calculating a plurality of utility functions based on the target ranges; and
calculating dynamically, by the processor, a single stability value associated with the patient based the target ranges of each physiological variable in relation to the estimated trends, wherein the stability value is a single value that reflects and accounts for the plurality of physiological variables to indicate the stability of the patient.

2. The method of claim 1, wherein calculating the estimated trend of each physiological variable includes:
performing a plurality of linear estimations over a number of time intervals for each physiological variable of a plurality of physiological variables associated with the patient that are acquired from the plurality of medical devices.

3. The method of claim 2, wherein a linear estimation is performed for each physiological variable received from a particular medical device over a first period of time that is subsequently parsed into a second period of time that is smaller than the first period of time.

4. The method of claim 3, wherein the second period of time is parsed additively and inclusively and wherein a length of time associated with the first period of time determines how much of a patient's history is included in the linear estimation, and a length of time of the second period of time determines how sensitive the stability value will be in relation to resulting trends.

5. The method of claim 3, wherein the first period of time and the second period of time are set prior to performing the linear estimation and are based at least on an input by a user and physiologic process time constraints.

6. The method of claim 3, further comprising:
generating a third time period back from a current time value when the stability of the patient is initially assessed before a certain amount of data and time value has been acquired, wherein the linear estimation is performed over the third time period during the third time period, and
once a sufficient amount of data and time values has been acquired,
looping each time value, and
calculating within each loop a dominate trend, a standard deviation and an estimated slope for each physiological variable of the plurality of physiological variables.

7. The method of claim 1, wherein for each physiological variable of the plurality of physiological variables over time, the trend calculation returns an estimated slope, a standard deviation and an estimation value of each physiological variable over given period of time.

8. The method of claim 1, wherein calculating the single stability value of the patient includes outputting an utility map estimate for each physiological variable by feeding the estimated trend of each physiological variable at the given point in time through a utility map to normalize each physiological variable relative to other physiological variables of the plurality of physiological variables and weight each of the normalized physiological variables depending on where each of the normalized physiological variables falls within the targeted range of that physiological variable,
wherein the target range for each physiological variable is set by a user on a user interface.

9. The method of claim 8, wherein the single stability value is calculated by multiplying a mean of the utility map estimates of each physiological variable by the estimated slope of each physiological variable to output a mean value for each physiological variable and a sum of the mean value for each physiological variable over the plurality of physiological variables.

10. The method of claim 8, wherein the single stability value is calculated by multiplying a mean of the utility map estimates of each physiological variable by the estimated slope of each physiological variable to output a max value for each physiological variable and summing the max value for each physiological variable over time.

11. The method of claim 8, wherein the single stability value is calculated by:
multiplying a mean of the utility map estimates of each physiological variable by the estimated slope of each physiological variable to output a resulting mean value;
multiplying the output resulting mean value by a normalized standard deviation value to output a weighted value for each physiological value; and
summing the weighted value for each physiological value over the plurality of physiological variables.

12. The method of claim 8, wherein the single stability value is calculated by:
multiplying a mean of the utility map estimates of each physiological variable by the estimated slope of each physiological variable to output a resulting mean value;
multiplying the outputted resulting mean value by a length of time associated with each trend to output a weighted value; and
summing the weighted value over the plurality of physiological variables.

13. The method of claim 1, wherein utility map data for each physiological variable is output prior to calculating estimated trends.

14. A non-transitory computer readable medium containing program instructions executed by a processor, the computer readable medium comprising:
program instructions that calculate a plurality of estimated trends for each physiological variable by utilizing a plurality of physiological variables from a plurality of medical devices and a plurality of target ranges for each physiological variable of the plurality of physiological variables over time;
program instructions that calculate a plurality of utility functions based on the target ranges; and
program instructions that calculate dynamically a single stability value associated with the patient based the target ranges of each physiological variable in relation to the estimated trends, wherein the stability value is a single value that reflects and accounts for the plurality of physiological variables to indicate the stability of the patient.

15. The non-transitory computer readable medium of claim 14, wherein the program instructions that calculate the estimated trend of each physiological variable includes:
program instructions that perform a plurality of linear estimations over a number of time intervals each physiological variable of a plurality of physiological variables associated with the patient that are acquired from a plurality of medical devices.

16. The non-transitory computer readable medium of claim 15, wherein a linear estimation is performed for each physiological variable received from a particular medical device over a first period of time that is subsequently parsed into a second period of time that is smaller than the first period of time.

17. The non-transitory computer readable medium of claim 16, wherein the second period of time is parsed additively and inclusively and wherein a length of time associated with the first period of time determines how much of a patient's history is included in the linear estimation, and a length of time of the second period of time determines how sensitive the stability value will be in relation to resulting trends.

18. The non-transitory computer readable medium of claim 16, further comprises:
program instructions generate a third time period back from a current time value when the stability of the patient is initially assessed before a certain amount of data and time values has been acquired, wherein the linear estimation is performed over the third time period, and
program instructions that loop each time value, and calculate within each loop a dominate trend, a standard deviation and an estimated slope for each physiological variable once a sufficient amount of data and time values has been acquired.

19. The non-transitory computer readable medium of claim 1, wherein for each physiological variable of the plurality of physiological variables over time, the programs instructions that estimate the trend for each physiological variable include program instructions that return an estimated slope, a standard deviation and an estimation value of each physiological variable over a given point in time.

20. The non-transitory computer readable medium of claim 14, wherein program instructions that calculate the single stability value of the patient includes outputting an utility map estimate for each physiological variable by feeding the estimated trend of each physiological variable at the given point in time through a utility map to normalize each physiological variable relative to other physiological variables of the plurality of physiological variables and weight each of the normalized physiological variables depending on where each of the normalized physiological variables falls within the targeted range of that physiological variable,
wherein the targeted range for each physiological variable is set by a user via a user interface.

21. The non-transitory computer readable medium of claim 20, wherein the single stability value is calculated by multiplying a mean of the utility map estimates of each physiological variable by the estimated slope of each physiological variable to output a mean value for each physiological variable and a sum of the mean value for each physiological variable over the plurality of physiological variables.

22. The non-transitory computer readable medium of claim 21, wherein the single stability value is calculated by multiplying a mean of the utility map estimates of each physiological variable by the estimated slope of each physiological variable to output a max value for each physiological variable and summing the max value for each physiological variable over time.

23. The non-transitory computer readable medium of claim 21, wherein the single stability value is calculated by:
program instructions that multiply a mean of the utility map estimates of each physiological variable by the estimated slope of each physiological variable to output a resulting mean value;
program instructions that multiply the output resulting mean value by a normalized standard deviation value to output a weighted value for each physiological value; and
program instructions that sum the weighted value for each physiological value over the plurality of physiological variables.

24. The non-transitory computer readable medium of claim 8, wherein the single stability value is calculated by:
program instructions that multiply a mean of the utility map estimates of each physiological variable by the estimated slope of each physiological variable to output a resulting mean value;
program instructions that multiply the outputted resulting mean value by a length of time associated with each trend to output a weighted value; and
program instructions that sum the weighted value over the plurality of physiological variables.

25. A system for assessing a stability of a patient, comprising:
a network adaptor configured to receive a plurality of physiological variables from a plurality of medical devices,
a memory configured to store a plurality of target ranges for each physiological variable of the plurality of physiological variables; and
a processor configured to calculate a plurality of estimated trends for each physiological variable over time, calculate a plurality of utility functions based on the target ranges; and calculate dynamically a single stability value associated with the patient based the target ranges of each physiological variable in relation to the estimated trends, wherein the stability value is a single value that reflects and accounts for the plurality of physiological variables to indicate the stability of the patient.

26. The system of claim 25, wherein the processor is further configured to:
perform a plurality of linear estimations over a number of time intervals for each physiological variable of a plurality of physiological variables associated with the patient that are acquired from the plurality of medical devices.

27. The method of claim 25, wherein for each physiological variable of the plurality of physiological variables over time, the trend calculation returns an estimated slope, a standard deviation and an estimation value of each physiological variable over a given point in time.

28. The system of claim 1, wherein calculating he single stability value of the patient includes outputting an utility map estimate for each physiological variable by feeding the estimated trend of each physiological variable at the given point in time through a utility map to normalize each physiological variable relative to other physiological variables of the plurality of physiological variables and weight each of the normalized physiological variables depending on where each of the normalized physiological variables falls within the targeted range of that physiological variable,
wherein the targeted range for each physiological variable is set by a user via a user interface.

29. The system of claim 28, wherein the processor is configured to calculate the single stability value by multiplying a mean of the utility map estimates of each physiological variable by the estimated slope of each physiological variable to output a mean value for each physiological variable and a sum of the mean value for each physiological variable over the plurality of physiological variables.

30. The system of claim 28, wherein the processor is configured to calculate the single stability value by multiplying a mean of the utility map estimates of each physiological variable by the estimated slope of each physiological variable to output a max value for each physiological variable and summing the max value for each physiological variable over time.

31. The system of claim 28, wherein the processor is configured to calculate the single stability value by:
multiplying a mean of the utility map estimates of each physiological variable by the estimated slope of each physiological variable to output a resulting mean value;
multiplying the output resulting mean value by a normalized standard deviation value to output a weighted value for each physiological value; and
summing the weighted value for each physiological value over the plurality of physiological variables.

32. The system of claim 28, wherein the processor is configured to calculate the single stability value by:
multiplying a mean of the utility map estimates of each physiological variable by the estimated slope of each physiological variable to output a resulting mean value;
multiplying the outputted resulting mean value by a length of time associated with each trend to output a weighted value; and
summing the weighted value over the plurality of physiological variables.
